(19) United States
(12) Patent Application Publication
(10) Pub. No.: US 2004/0126445 A1
(43) Pub. Date: Jul. 1, 2004

(54) Laurel based healing composition

(75) Inventor: Shimon Getta, Kfar Yona (IL)

Correspondence Address: DR. MARK FRIEDMAN LTD., C/o Bill Polkinghorn, Discovery Dispatch, 9003 Florin Way, Upper Marlboro, MD, 20772, US

(73) Assignee: ELIYAHU SAMILA

(21) Appl. No.: 10/331,544

(22) Filed: Dec. 31, 2002

(51) Int. Cl. A61K 35/78

(52) U.S. Cl. 424/769

(57) ABSTRACT

A healing composition with a broad-spectrum sanative effect, substantially consisting of laurel extract in wine and especially a wine laurel extract, is disclosed.

### FIELD OF THE INVENTION

The present invention relates generally to therapeutic compositions and more specifically to a composition for the relief of a broad spectrum of skin conditions.

### BACKGROUND OF THE INVENTION

Many plants produce chemicals as part of a defense against environmental challenges encountered by the plant in a specific biotope. Such chemicals often have, for example, antimycotic, antibacterial, antiparasitic, insectifugal and insecticidal properties. Although rarely is any one chemical very concentrated in a plant, the synergistic effect of many tens or hundreds of individual chemical compounds in specific ratios allows plants to mount a highly successful chemical defense against environmental challenges encountered. Herein the chemical arsenal of a plant will be termed a pharmacopoeia.

Plant pharmacopoeia are used for the treatment and prevention of ailments. Originally the use of the medicinal properties of the natural chemical defenses of plants was through consumption of plant parts or through the direct application of sap from bruised or otherwise damaged plant parts. With time the concept of increasing the efficacy of plant-based medicine by concentrating the essence of the plant gained legitimacy. Water extractions (teas and infusions), alcoholic extractions (tinctures) and distillation of extracts (oils) are all popular ways of concentrating plant pharmacopoeia. Also popular for external use are ointments made by boiling powdered plant products in an oil or fat. In more recent times extraction methods have been developed using organic solvents (e.g. diethyl ether, chloroform) or physical methods such as steam distillation.

All these approaches are based on the mistaken assumption that there exists a correlation between concentration and relative efficacy. Clearly, the amounts and types of chemicals in a plant pharmacopoeia have evolved to be effective within the specific chemical milieu found within that plant to face specific challenges. When extracted from the plant for use by humans, everything is different:

a. the challenges a plant faces are not the challenges faced by a human user;

b. the physical environment (e.g. concentrations, salinity, pH, oxygen and osmotic pressure) inside a plant is not the physical environment of an extract during preparation or when used as a medicament; and

c. the relative concentrations of chemicals in a pharmacopoeia change due to differences in solubility in the extraction solvent or due to the destruction of plant membranes.

These factors change the effectiveness of an individual component and often the chemical nature of a component. The effect of these factors is multiplied since the use of a plant pharmacopoeia is generally effective due to the synergistic effect of the plurality of components therein, rather than the existence of a single “magic bullet” compound.

It must therefore be concluded that it is impossible to know, a priori, the ideal method of utilizing a given plant pharmacopoeia. A specific method of extracting and using such a pharmacopoeia may be effective for one plant but not for a different plant. The discovery of a successful method of production of a plant-based composition is by definition serendipitous.

One plant known in the art for having medicinal value is the laurel (laurus nobilis), a member of the Lauraceae family also known as bay leaf. The laurel is an evergreen shrub or tree, indigenous to the Mediterranean region. Fresh leaves of the laurel typically contain between 1% and 3% oil of laurel with maximal oil content in mid-summer. Oil of laurel includes 1,8-cineole, eugenol, eugenol acetate, geraniol, 1-linalool, methyleugenol, phellandrene, pinene, sabinene, 1-terpineol acetate, esters and terpenoids.

Laurel is a highly popular flavoring agent both for pickled comestibles, cooked foods (e.g. Spanish patent ES 2 009 679) and alcoholic beverages (e.g. the famed Danish beverage “Gammel Dansk” or as described in Russian patent RU 2 120 966).

Both laurel ointment (oil or fat extraction) and laurel oil (cold pressed or from steam distillation) are suggested for external use to treat ear pain, arthritis, rheumatism, sprains and skin problems or as insect repellents.

Laurel leaves and berries (as infusions or powdered in capsules or water solutions) are suggested as amenorrhetics, anticolics, astringents, diaphoretics, stimulants, emetics and carminatives. Laurel berries have been used as abortifacients.

U.K. Patent Application GB 2 140 685 discloses the use of laurel oil combined with finely ground glass to treat alopecia.

Laurel, as used in the prior art, may also be harmful. Allergy to laurel oil has been reported by Hausen (Dtsch. Med. Wochenschr. 1985, 110(16), 634-638), Larregue et al. (Ann. Dermatol. Venereol. 1978, 105(5), 547-548), Foussereau et al. (Contact Dermatitis 1975, 1(4), 223-230) and Özden et al. (Contact Dermatitis 2001, 45(3), 178).

Despite the promise of using the laurel pharmacopoeia as a basis for a composition to alleviate medical indications, the most advantageous method of preparing such a composition is unknown. There is a need for a method for preparing an effective composition from the laurel plant.

### SUMMARY OF THE INVENTION

The above and other objectives are achieved by the method of preparing a composition of the present invention. Further, the composition prepared by the method of the present invention is exceptionally effective in alleviating discomfort and accelerating the healing of a broad spectrum of skin ailments. Further, the present invention teaches new and highly effective methods of treating a broad spectrum of conditions, both medical ailments and cosmetic conditions. By treating, herein is meant both alleviating symptoms associated with an ailment, accelerating healing of the ailment itself, or a cosmetic improvement.

According to the teachings of the present invention there is provided a method of preparing a composition comprising providing laurel parts and making a mixture of an amount of the laurel parts with an amount of a first aqueous solution so as to extract useful and effective substances making up the pharmacopoeia of laurel from the laurel parts, wherein the first aqueous solution includes sugars and more than about 5% alcohol by volume. The volume of the amount of the first aqueous solution added is preferably between about 25% and about 400% of the volume of the amount of laurel parts and more preferably between about 50% and about 200% of the volume of the amount of laurel parts.

According to a feature of the present invention, the extraction is performed at a temperature of less than about 50° C., more preferably less than about 40° C., and even more preferably less than about 30° C.

According to a feature of the present invention, after sufficient time has elapsed (preferably longer than an hour, more preferably longer than a day, even more preferably longer than about 5 days) the mixture is filtered so as to isolate a cake and a filtrate, the filtrate being the composition of the present invention.

According to a further feature of the present invention, after sufficient time has elapsed (preferably longer than an hour, more preferably longer than a day, even more preferably longer than about 5 days) the mixture is filtered so as to isolate a cake and a filtrate, and a second mixture is made by mixing an amount of the cake with an amount of a second aqueous solution so as to extract useful and effective substances making up the pharmacopoeia of laurel from the cake, wherein the second aqueous solution includes sugars and more than about 5% alcohol by volume.

The extraction of the cake using the second aqueous solution occurs over a time period that is preferably longer than an hour, more preferably longer than a day, even more preferably longer than about 5 days. Thereafter, the second mixture is filtered so as to isolate a second filtrate as the composition of the present invention. The volume of the amount of the second aqueous solution added is preferably between about 25% and about 400% of the volume of the amount of cake and more preferably between about 50% and about 200% of the volume of the amount of cake.

Generally, but not necessarily, the first and second aqueous solutions are substantially similar. According to a feature of the present invention, the aqueous solutions includes more than about 5%, preferably more than about 8% and even more preferably more than about 10% alcohol by volume. According to a further feature of the present invention the aqueous solutions includes more than about 2% and preferably more than about 5% sugars by weight. Preferably either or both the first and second aqueous solutions are wine, preferably red wine, more preferably sweet red wine.

There is also provided according to the teachings of the present invention a composition comprising an aqueous solution and extract of laurel, wherein the aqueous solution includes sugars and more than 5% alcohol by volume.

Preferably, the extract of laurel is of ground laurel leaves, twigs and bark extracted with a sugar/alcohol aqueous solution, especially wine.

Most preferably the extract of laurel is an extract of laurel, substantially prepared in accordance with the method of the present invention.

According to a feature of the present invention, the aqueous solution from which the composition of the present invention is made includes more than about 5%, preferably more than about 8% and even more preferably more than about 10% alcohol by volume.

According to a further feature of the present invention the aqueous solution from which the composition of the present invention is made includes more than about 2% and preferably more than about 5% sugars by weight.

Preferably the aqueous solution from which the composition of the present invention is made is wine, preferably red wine, more preferably sweet red wine.

There is also provided according to the teachings of the present invention a method of treating a cosmetic condition by topical application of a composition of the present invention. The cosmetic conditions treatable according to the teachings of the present invention include dry skin, chapped skin, callused skin and acne vulgaris.

There is also provided according to the teachings of the present invention a method of treating an ailment by topical application of a composition of the present invention. The ailments treatable according to the teachings of the present invention include insect bites, insect stings, burns, abscess, polyps, dry skin, chapped skin, callused skin, acne vulgaris, fungal infections, tinea pedis, skin wounds, arthritis, rheumatism, pain, inflammation, insect infestation, swelling and sprains.

There is also provided according to the teachings of the present invention for the use of a composition of the present invention for the manufacture of a medicament, such as an analgesic, anti-inflammatory, antibacterial, antiseptic, moisturizing, antipruritic, antimycotic, insecticide, insectifuge, astringent, cleansing, emollient, sanative, disinfectant and antiedemic medicament.

There is also provided according to the teachings of the present invention for the use of a composition of the present invention for the manufacture of a medicament for the treatment of a conditions such as insect bites, stings, burns, abscess, polyps, dry skin, chapped skin, callused skin, acne vulgaris, fungal infections, tinea pedis, skin wounds, arthritis, rheumatism and sprains.

### DETAILED DESCRIPTION OF THE INVENTION

The present invention is of a method of preparing a composition, a composition and a method of treating skin ailments and cosmetic problems using the composition.

Method of Preparation

Most generally, the method of preparing a composition of the present invention can be considered the extraction of substances of the laurel pharmacopoeia from laurel plant parts using wine (or wine imitation) as an extraction solvent.

According to the method of the present invention, extraction involves mixing the plant parts with an extraction solution and letting extraction occur over time. Preferably, to a given volume of laurel parts between about 25% and about 400% volume, more preferably between about 50% and about 200% volume, extraction solvent is added.

Although any part of the laurel can be used, preferably leaves, bark and twigs are used. The laurel parts are finely ground or crushed.

Although any aqueous alcohol/sugar solution can be used, wine is the preferred extraction solvent. Although any wine can be used, red wine is preferred.

Although wine with any alcohol content can be used, a wine having an alcohol content of greater than about 5%, more preferably greater than about 8%, and even more preferably greater than about 10% by volume.

Although wine with any sugar content can be used, preferred is a wine having greater than about 2% sugar content by weight (sweeter than semi dry) and more preferred is a wine having greater than about 5% sugar content by weight (sweeter than semi sweet).

In conclusion, according to the method of the present invention most preferable is a sweet red wine.

As is clear to one skilled in the art, wine is substantially sugar in a water/ethanol solution. It is likely that some of the advantages of the present invention can be mimicked by using an aqueous solution of sugar and alcohol instead of wine, where the relative amounts of sugar and alcohol in the solution are chosen so as to resemble those of wine. Similarly, instead of ethanol another alcohol may be used, especially isopropanol.

According to the present invention, extraction is preferably performed under moderate conditions, at a temperature no higher than 50° C., preferably less than 40° C., more preferably less than 30° C.

According to the present invention, extraction is allowed to proceed for a relatively long time, preferably longer than an hour, more preferably longer than a day, even more preferably longer than about 5 days.

It has been found that a second extraction of a once extracted portion of laurel parts yields an exceptionally effective composition.

Composition

Most generally, the composition of the present invention can be considered as an extract of laurel in wine, especially when made in accordance with the method of the present invention. The composition of the present invention is somewhat more viscous than water being substantially an oil in a sugar/water/ethanol solution, known as wine.

As stated above, although any aqueous alcohol/sugar solution can be used, wine is the preferred extraction solvent. Although any wine can be used, red wine is preferred.

Although wine with any alcohol content can be used, a wine having an alcohol content of greater than about 5%, more preferably greater than about 8%, and even more preferably greater than about 10% by volume.

Although wine with any sugar content can be used, preferred is a wine having greater than about 2% sugar content by weight (sweeter than semi dry) and more preferred is a wine having greater than about 5% sugar content by weight (sweeter than semi sweet).

In conclusion, the preferred embodiment of a composition of the present invention is a wine extract of laurel in a sweet red wine.

It is important to note that it is likely that a prior art laurel oil mixed with wine has improved healing properties even though the ratio and concentration are different from the compositions produced by the method of the present invention.

It is important to note that it is likely that a prior art laurel oil mixed with a water/alcohol/sugar solution having a composition resembling that of wine may have improved healing properties. As is clear to one skilled in the art, wine is substantially sugar in a water/ethanol solution. It is likely that some of the advantages of the present invention can be mimicked by using an aqueous solution of sugar and alcohol instead of wine, where the relative amounts of sugar and alcohol in the solution are chosen so as to resemble those of wine. Similarly, instead of ethanol another alcohol may be used, especially isopropanol.

Thus although considered to be less effective, obvious variants of the present invention such as the two discussed immediately hereinabove are considered to be part of the present invention.

Method of Treatment

Most generally, the method of treating according to the present invention includes the topical application of the composition of the present invention to treat a medical ailment or a cosmetic problem.

The primary properties of the composition of the present invention are relief of itching and pain of the skin when topically applied. The composition of the present invention also promotes the natural healing process of the body itself. The composition of the present invention also either stops the growth or kills bacteria, fungus and insects (scabies mites). The composition treats and relieves such non-medical cosmetic problems as acne and dryness of the skin.

Two methods of applying the composition of the present invention have been tested. The first and apparently more effective method has been found to be by providing a dressing, wet with the composition of the present invention, in continuous contact with an area to be treated. The dressing is substantially maintained wet with the composition. The second method is smearing or wiping of an amount of the composition of the present invention on an area to be treated using the finger or an applicator such as a cotton swab.

Although not wishing to be held to any theory, it is believed that the exceptional therapeutic effects of the present invention are attributable to two reasons. First, the extraction solvent being an alcohol/sugar/water solution is exceptionally suited for extracting the active compounds of the laurel pharmacopoeia in concentrations and ratios that are suitable for use in treating human skin ailments. Second, the effect of the compounds extracted from laurel using wine are apparently exceptionally effective in the presence of sugars (known bacteriostats and nutrients) and other compounds found in wine (e.g. anthocyanins in red wine).

The effectiveness of the composition of the present invention illustrated by a wealth of clinical results. The following case histories illustrate the overall effectiveness of the present invention.

### EXPERIMENTAL RESULTS

Preparation of a First Composition of the Present Invention

Fresh leaves and twigs of up to 1 cm in diameter were gathered and ground to small pieces. The small pieces were crushed using a stone mortar and pestle. A volume of crushed pieces was transferred to a bottle. To the bottle was added an amount of sweet red wine having 14% alcohol (“Conditon” by Hacormim Wine Cellars, Mishor Edumim, Israel) so as to double the amount of material in the bottle. That is, to 100 ml crushed pieces, wine was added so that the total volume of material in the bottle was 200 ml. The bottle was sealed, agitated and stored at 15° C. in a dark room. After a week, the contents of the bottle were filtered through a fine meshed cotton cloth and the filter cake manually wrung. The liquid filtrate, a yellowish brown liquid was bottled and stored for use as the first solution.

Preparation of a Second Solution of the Present Invention

The filter cake, above, was transferred to a bottle and an additional volume of sweet red wine was added. To the bottle was added an amount of sweet red wine having 14% alcohol (“Conditon” by Hacormim Wine Cellars, Mishor Edumim, Israel) so as to increase the total amount of material in the bottle by half. That is, to 100 ml filter cake, wine was added so that the total volume of material in the bottle was 150 ml. The bottle was sealed, agitated and stored at 15° C. in a dark room. After a week, the contents of the bottle were filtered through a fine meshed cotton cloth and the filter cake manually wrung. The liquid filtrate, a yellowish brown liquid was bottled and stored for use as the second solution.

### Cinical Results

### EXAMPLE 1

### Burn Wound (Analgesic, Antiinflammatory, Sanative)

A 1 cm² area of the finger of a 44 year old male was burnt by contact with boiling oil. A cotton dressing soaked with the first solution was kept in contact with the burn area. Periodically, when the dressing was observed to be dry, additional solution was added. Immediate and continuous relief from pain was observed. A non-painful blister developed in the area of the wound that eventually opened and drained. The opened blister healed leaving no noticeable scar.

### EXAMPLE 2

### Abscess (Antibacterial, Antiseptic, Sanative)

A 37 year old male was bedridden after a serious motorcycle accident. Pressure wounds developed on the back of the patient and became infected, leading to formation of a plurality of abscesses. A cotton dressing soaked with the first solution was kept in contact with each abscess. Immediate and continuous relief from pain was observed. The dressings were replaced twice daily. After 10 days the abscesses had healed.

### EXAMPLE 3

### Polyp

A skin polyp developed on the finger of a 35 year old female. A cotton dressing soaked with the first solution was kept in contact with the polyp. It was observed that the polyp degenerated until complete disappearance after two weeks.

### EXAMPLE 4

### Callused and Dry Skin (Emollient, Sanative)

A number of patients reported callused and dry skin of the hands. The patients were given a first solution with which to smear the hands when desired. All patients reported relief and healing of the skin, including disappearance of previously present cracks in the skin.

### EXAMPLE 5

### Aspecific Dermal Irritation (Antipruritic, Antiinflammatory, Emollient, Sanative)

A 35-year old male had chronic aspecific dermal irritation characterized by the occasional appearance of a red rash followed by peeling and cracking of the skin. Previous treatment with hydrocortisone containing creams was ineffective. Twice daily treatment with the first solution caused the rash to disappear softened the skin and gave relief from the peeling.

### EXAMPLE 6

### Acne Vulgaris (Antibacterial, Astringent, Cleansing)

A 17 year old female and a 22 year old female both had extensive appearance and reappearance of acne pustules on the face. Both were asked to treat such pustules when appearing by applying the first solution using a cotton swab. Both patients reported that pustules healed quickly and a significant improvement in the general appearance of the skin.

### EXAMPLE 7

### Tinea Pedis (Antimycotic, Antipruritic)

A 44 year old male with a tinea pedis infection applied the first solution twice daily to the affected area, or whenever itching became unbearable. Immediate relief from itching was observed. After two weeks the feet appeared clear of the tinea pedis infection.

### EXAMPLE 8

### Nail Fungus (Antimycotic)

A 44 year old female had a finger nail chronically infected with fungus for 12 years. At least twice daily application of the first solution by dipping the fingernail resolved the infection. New nail growth was observed to be healthy.

### EXAMPLE 9

### Scabies (Antipruritic, Insecticide, Insectifuge, Sanative)

Five patients, both male and female were diagnosed as having a scabies parasitic infection. The patients were instructed to treat all infected areas multiple times daily, whenever itching became apparent, by wiping with a cloth containing the first solution. Immediate relief from itching and disappearance of the scabies after between 2 and 3 weeks was observed.

### EXAMPLE 10

### Insect Bite (Antipruritic, Antiedemic, Antiinflammatory)

Numerous children of both sexes and of a wide age distribution were bitten by mosquitoes. The area of the bite was swollen, red and itchy. An amount of the first solution was applied to the area using a cotton swab. Immediate relief from itching was observed.

### EXAMPLE 11

### Insect Sting (Antipruritic, Analgesic, Antiedemic, Antiinflammatory)

A 12 year old male was stung by a bee. The area of the sting was swollen, red and very painful. No evidence of an allergic reaction was observed. A cotton dressing soaked with the first solution was kept in contact with the swollen area. Immediate relief and reduction of swelling was observed.

A 8 year old female was stung by a wasp. The area of the sting was swollen, red and very painful. No evidence of an allergic reaction was observed. A cotton dressing soaked with the first solution was kept in contact with the swollen area. Immediate relief and reduction of swelling was observed.

### EXAMPLE 12

### Swollen Feet (Antiedemic)

A 35 year old woman and a 40 year old woman walked extensively in an urban environment. As a result both had painfully swollen feet. Both smeared the feet with a generous amount of the first solution. Immediate relief from pain followed by relatively quick reduction of swelling was observed.

### EXAMPLE 13

### Superficial Skin Wounds (Disinfectant, Sanative)

A 5 year old female struck her head on the corner of a table, causing a large but superficial cut on the scalp. The first solution was applied to the cut 3-4 times a week with the help of a dressing. The wound quickly healed with little pain and no infection. After a week only a small, virtually invisible, scar remained.

From the known properties of laurel it is expected that that the composition of the present invention is also useful in treating ear pain, arthritis, rheumatism, sprains and skin problems or as an insect repellent.

The present invention is not limited to the embodiments described herein but encompasses any and all embodiments a within the scope of the claims, including obvious variants thereof. Such obvious variants include replacing wine with aqueous solutions of sugars and alcohols.

1. A method of preparing a composition comprising:
a) providing laurel parts; and
b) making a mixture of an amount of said laurel parts with an amount of a first aqueous solution so as to extract substances from said laurel parts
wherein said first aqueous solution includes sugars and more than about 5% alcohol by volume.

2. The method of claim 1 wherein said extracting is performed at a temperature of less than about 50° C.

3. The method of claim 1 further comprising:
c) filtering said mixture so as to isolate a cake and a filtrate; and
d) isolating said filtrate as the composition.

4. The method of claim 1 further comprising:
c) filtering said mixture so as to isolate a cake and a filtrate.
d) making a second mixture by mixing an amount of said cake with an amount of a second aqueous solution so as to extract substances from said cake
wherein said second aqueous solution includes sugars and more than about 5% alcohol by volume.

5. The method of claim 1 wherein said amount of said first aqueous solution added has a volume of between about 25% and about 400% of a volume of said amount of said laurel parts.

6. The method of claim 1 wherein said amount of said first aqueous solution added has a volume of between about 50% and about 200% of a volume of said amount of said laurel parts.

7. The method of claim 4 wherein said amount of said second aqueous solution added has a volume of between about 25% and about 400% of a volume of said amount of said cake.

8. The method of claim 4 wherein said amount of said second aqueous solution added has a volume of between about 50% and about 200% of a volume of said amount of said cake.

9. The method of claim 1 wherein said first aqueous solution is wine.

10. The method of claim 1 wherein said second aqueous solution is wine.

11. A composition comprising:
a) an aqueous solution; and
b) extract of laurel
wherein said aqueous solution includes sugars and more than 5% alcohol by volume.

12. The composition of claim 11 wherein said extract of laurel is a wine extract of laurel.

13. The composition of claim 11 wherein said aqueous solution is wine.

14. The composition of claim 13 wherein said wine is red wine.

15. The composition of claim 13 wherein said wine has a sugar content greater than 2% by weight.

16. The composition of claim 11 wherein said aqueous solution includes more than about 8% alcohol by volume.

17. The composition of claim 11 wherein said aqueous solution includes more than 2% sugars by weight.

18. A method of treatment comprising topical application of a composition according to claim 11.

19. The method of claim 18 wherein said composition is for the treatment of a cosmetic condition selected from the group of conditions consisting of dry skin, chapped skin, callused skin and acne vulgaris.

20. The method of claim 18 wherein said composition is for the treatment of an ailment selected from the group of conditions consisting of insect bites, insect stings, burns, abscess, polyps, dry skin, chapped skin, callused skin, acne vulgaris, fungal infections, tinea pedis, skin wounds, arthritis, rheumatism, pain, inflammation, insect infestation, swelling and sprains.

21. Use of the composition according to claim 11 for the manufacture of a medicament.

22. The use of claim 21 wherein said medicament is selected from the group consisting of analgesic, anti-inflammatory, antibacterial, antiseptic, moisturizing, antipruritic, antimycotic, insecticide, insectifuge, astringent, cleansing, emollient, sanative, disinfectant and antiedemic medicaments.

23. The use of claim 21 wherein said medicament is for the treatment of a condition selected from the group of conditions consisting of insect bites, stings, burns, abscess, polyps, dry skin, chapped skin, callused skin, acne vulgaris, fungal infections, tinea pedis, skin wounds, arthritis, rheumatism and sprains.
